(19) United States
(12) Patent Application Publication
(10) Pub. No.: US 2011/0077503 A1
(43) Pub. Date: Mar. 31, 2011

(54) Automatic MRI Quantification of Structural Body Abnormalities

(75) Inventors: Leonardo Bonilha, Charleston, SC (US); Jonathan Edwards, Mt. Pleasant, SC (US)

(73) Assignee: MEDICAL UNIVERSITY OF SOUTH CAROLINA, Charleston, SC (US)

(21) Appl. No.: 12/868,613

(22) Filed: Aug. 25, 2010

Related U.S. Application Data
(60) Provisional application No. 61/236,734, filed on Aug. 25, 2009.

(51) Int. Cl. A61B 5/055; G06K 9/00

(52) U.S. Cl. 600/411; 382/131

(57) ABSTRACT

Disclosed herein are methods and systems related to the body region analysis. In some forms, the analysis relates to body region abnormalities. In some forms, the methods and systems analyze and assign comparison scores (such as Z-score) to an image of a body region. In some forms, the comparison score is plotted voxel-by-voxel. In some forms, the computers and systems are adapted to execute the methods disclosed herein

### CROSS-REFERENCE TO RELATED APPLICATIONS

This application claims benefit of U.S. Provisional Application No. 61/236,734, filed Aug. 25, 2009, and is hereby incorporated by reference in its entirety

### BACKGROUND

Detection of brain abnormalities can be done using imaging techniques such as but not limited to electron microscopy, radiographic methods, magnetic resonance imaging (MRI), nuclear medicine, photoacoustic methods, thermal methods, tomography, ultrasound, computed axial tomography, diffuse optical imaging, event-related optical signal, functional magnetic resonance imaging, magnetoencephalography, positron emission tomography, single photon emission computed tomography and other modalities. Imaging methodologies typically will generate an image that can be examined visually by a clinician and additionally may lend itself to analysis using automated methods often commonly called image processing. Image processing can involve the manipulation of digital images to provide clarification or enhancement of regions or interest. Further, image processing can be used to perform statistical and other mathematical techniques upon image(s) to aid the clinician in understanding function and diagnosing illness.

### SUMMARY

Disclosed herein are methods and systems related to the body region analysis. In some forms, the analysis relates to body region abnormalities.

In some forms, the methods and systems analyze and assign comparison scores (such as Z scores) to an image of a body region. In some forms, the comparison scores is plotted voxel-by-voxel. In some forms, the computers and systems are adapted to execute the methods disclosed herein.

### BRIEF DESCRIPTION OF THE DRAWINGS

FIG. 1A shows on panel ‘A’, the first row demonstrates the non-smoothed voxel wise mean of gray matter within control subjects, while the second row shows the voxel wise standard deviation of gray matter in controls. The hippocampal region (highlighted with a white frame) shows a high mean value of gray matter with a relative low standard deviation. The deviations from the normal distribution are identifiable through voxel Z score maps, as demonstrated in 1B on panel ‘B’, showing the location of Z score <−3.5 in two representative subjects, illustrating left (upper row) and right (bottom row) MTLE.

FIG. 2 shows the distribution of the mean voxel-wise Z scores within the hippocampi is demonstrated by percentile histograms on the left panel (A). Each hippocampus is represented just once, and frequency is plotted against the mean Z score of the lowest 25% Z scores within the hippocampi. The inlet demonstrates the values of the fitted ROC curves comparing different mean values of the lowest percentiles of Z scores. Note the peak of fitted ROC value (0.973) for the lowest 25%. The right panel shows the ROC comparing the ipsilateral hippocampi and hippocampi from control subjects. There is a high relative true positive rate (TPR) for low false positive rates (FPR=1−specificity).

### DETAILED DESCRIPTION

### A. Definitions

1. A, an, the

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

2. Body Region

Body region or the like terms refer to a defined part of the body. For example, a body region can be the brain or the brain region. A brain region can be a defined part of the brain, for example the hippocampus. A body region can also be for example, bones region, heart region, vascular system region, lung region, liver region, kidney region and intestine region. Any region can be a defined part within that region, for example, bone region can the femur or the vertebrae.

3. Body Region Abnormality

An abnormality or a body region abnormality or the like terms refers to something or a process of something that is different from what is commonly known or statistically determined to be normal. For example, a body region abnormality can be atrophy, hypertrophy, spatial deviation or changes in it structure. A body region abnormality can be determined statistically by comparing it to a population standard. A body region abnormality can for example be determined by a analyzing an image of a body region using an algorithm.

4. Clinical Concern

A clinical concern is a concern that something is abnormal in a subject. A clinical concern is not a diagnosis or prognosis but rather a suspicion that something could be abnormal in the subject. A body region abnormality can be, for example, a clinical concern.

5. Comprise

Throughout the description and claims of this specification, the word “comprise” and variations of the word, such as “comprising” and “comprises,” means “including but not limited to,” and is not intended to exclude, for example, other additives, components, integers or steps.

6. Computer Readable Media, Computer Program Product, Processors. Computer Usable Memory, Computer Systems

In some embodiments, instructions stored on one or more computer readable media that, when executed by a system processor, cause the system processor to perform the methods described above, and in greater detail below. Further, some embodiments can include systems implementing such methods in hardware and/or software. A typical system can include a system processor comprising one or more processing elements in communication with a system data store (SDS) comprising one or more storage elements. The system processor can be programmed and/or adapted to perform the functionality described herein. The system can include one or more input devices for receiving input from users and/or software applications. The system can include one or more output devices for presenting output to users and/or software applications. In some embodiments, the output devices can include a monitor capable of displaying to a user graphical representation of the described analytic functionality.

The described functionality can be supported using a computer including a suitable system processor including one or more processing elements such as a CELERON, PENTIUM, XEON, CORE 2 DUO or CORE 2 QUAD class microprocessor (Intel Corp., Santa Clara, Calif.) or SEMPRON, PHENOM, OPTERON, ATHLON X2 or ATHLON 64 X2 (AMD Corp., Sunnyvale, Calif.), although other general purpose processors could be used. In some embodiments, the functionality, as further described below, can be distributed across multiple processing elements. The term processing element can refer to (1) a process running on a particular piece, or across particular pieces, of hardware, (2) a particular piece of hardware, or either (1) or (2) as the context allows. Some implementations can include one or more limited special purpose processors such as a digital signal processor (DSP), application specific integrated circuits (ASIC) or a field programmable gate arrays (FPGA). Further, some implementations can use combinations of general purpose and special purpose processors.

The environment further includes a system data store (SDS) that could include a variety of primary and secondary storage elements. In one preferred implementation, the SDS would include registers and RAM as part of the primary storage. The primary storage can in some implementations include other forms of memory such as cache memory, non-volatile memory (e.g., FLASH, ROM, EPROM, etc.), etc. The SDS can also include secondary storage including single, multiple and/or varied servers and storage elements. For example, the SDS can use internal storage devices connected to the system processor. In implementations where a single processing element supports all of the functionality a local hard disk drive can serve as the secondary storage of the SDS, and a disk operating system executing on such a single processing element can act as a data server receiving and servicing data requests.

It will be understood by those skilled in the art that the different information used in the systems and methods for respiratory analysis as disclosed herein can be logically or physically segregated within a single device serving as secondary storage for the SDS; multiple related data stores accessible through a unified management system, which together serve as the SDS; or multiple independent data stores individually accessible through disparate management systems, which can in some implementations be collectively viewed as the SDS. The various storage elements that comprise the physical architecture of the SDS can be centrally located or distributed across a variety of diverse locations.

7. Computer Network

A computer network or like terms are one or more computers in operable communication with each other.

8. Computer Implemented

Computer implemented or like terms refers to one or more steps being actions being performed by a computer, computer system, or computer network.

9. Computer Program Product

A computer program product or like terms refers to product which can be implemented and used on a computer, such as software.

10. Control

The terms “control” or “control levels” or “control cells” are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard.

11. Higher

The terms “higher,” “increases,” “elevates,” or “elevation” or variants of these terms, refer to increases above basal levels, e.g., as compared to a control or average values observed in the normal population. The terms “low,” “lower,” “reduces,” or “reduction” or variation of these terms, refer to decreases below basal levels, e.g., as compared to a control or average values observed in the normal population. For example, basal levels are normal in vivo levels prior to, or in the absence of, or addition of an agent such as an agonist or antagonist to activity.

12. Imaging Instrument

An imaging instrument is machine, apparatus or process that depicts objects or substances. An imaging instrument can for example be to electron microscopy, radiographic methods, magnetic resonance imaging (MRI), nuclear medicine, photoacoustic methods, thermal methods, tomography, ultrasound, computed axial tomography, diffuse optical imaging, event-related optical signal, functional magnetic resonance imaging, magnetoencephalography, positron emission tomography or single photon emission computed tomography.

13. Obtaining

Obtaining as used in the context of data or values, such as Z-score values or values refers to acquiring this data or values. It can be acquired, by for example, collection, such as through a machine, such as an imaging instrument. It can also be acquired by downloading or getting data that has already been collected, and for example, stored in a way in which it can be retrieved at a later time.

14. Optional

“Optional” or “optionally” means that the subsequently described event or circumstance may or may not occur, and that the description includes instances where said event or circumstance occurs and instances where it does not.

15. Outputting Results

Outputting or like terms means an analytical result after processing data by an algorithm.

16. Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data are provided in a number of different formats, and that this data, represents endpoints and starting points, and ranges for any combination of the data points. For example, if a particular datum point “10” and a particular datum point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

17. Reduce

By “reduce” or other forms of reduce means lowering of an event or characteristic. It is understood that this is typically in relation to some standard or expected value, in other words it is relative, but that it is not always necessary for the standard or relative value to be referred to. For example, “reduces phosphorylation” means lowering the amount of phosphorylation that takes place relative to a standard or a control.

18. Subject

“Subject” like terms refer to an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) and mammals, non-human mammals, primates, non-human primates, rodents, birds, reptiles, amphibians, fish, and any other animal. In one aspect, the subject is a mammal such as a primate or a human. The subject can be a non-human.

19. Treating

Treating” or “treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease. In certain embodiments, a treatment can actually do unforeseen harm to a subject.

20. Comparison Score.

Comparison score refers to a comparison of something to the population. For example, a body structure of a subject can be considered abnormal if its image is significantly different from the image of that structure in the average population. The comparison between the structure on that subject and the population can be performed by computing the difference between the subject and the population. Examples of comparison scores can be, but are not limited to, Z scores, ratios, or comparing the simple difference between the population average (or median) and that subject. In general, any mathematical calculation that reflects how different a subject is from the population standard can be used as a comparison score.

21. Z Score

The term “Z score” refers to how many standard deviations (from the general population) away from the mean (of the general population) the structure of that subject is. The Z score indicates a comparison to the average population.

22. Clinically Significant Difference

A clinically significant difference is defined by the clinician assessing the image. The difference may range from a very significant deviation or from a mild deviation depending on the structure and the clinical condition being assessed. Furthermore, the population standard against which the subject is being compared can encompass only normal subjects or another group that the clinician would like to compare that subject against.

23. Population Standard

A population standard or the like terms refer to the range, average or median of the image characteristics encountered in a body part in a population. A population standard can reflect a national, regional, local or specific database or a combination thereof. For example, the population standard can arise from a national database or come from the population that underwent a particular imaging instrument such as a MRI. The population standard can be limited to a specific population. For example, population standard can be the average signal for a body region for a specific population. The population can be based on age, gender, race, weight, height, geographical inhabitance or pervious diagnosis of a disease.

### B. Brain Region Abnormalities

Hippocampal sclerosis (HS) is the most common histological abnormality observed in patients with medial temporal lobe epilepsy (MTLE) (Margerison and Corsellis, 1966). It was first described in 1880 by Sommer (Sommer, 1880), and it is defined by segmental loss of pyramidal neurons, granule cell dispersion and reactive gliosis affecting mainly the CA1 and CA4 hippocampal regions (Blumcke et al., 2002).

HS is frequently associated with visible hippocampal atrophy on T1 weighted images and T2 signal hyperintensity on clinical Magnetic Resonance Imaging (MRI). However, visual inspection can miss these features in cases of mild or bilateral HS. Manually measuring hippocampal volume can improve sensitivity, detecting atrophy in 75 to 90% of the hippocampi on the side congruent with EEG seizure onset (Cendes et al., 1993; Jack et al., 1990). However, manual morphometry of the hippocampus is usually a tedious process, particularly with newer high-resolution MRI protocols that deliver thinner slices. Furthermore, the manual delineation of the anatomical boundaries of the hippocampus relies on the subjective judgment of anatomical landmarks, requiring specific training and possibly leading to rater dependent bias. These are important limitations that have precluded the use of manual morphometry for routine clinical practice. Disclosed herein are methods and systems related to automated MRI morphometry which can provide rapid, unbiased and accurate detection of hippocampal atrophy in subjects with MTLE. Such a technique can allow an objective measure of HS that could assist the neuroradiologist on the decision about the likelihood of hippocampal atrophy. The methods and systems can also be used for other clinical evaluations and techniques such as, for example, scores of bone density used to detect the presence of osteoporosis.

### C. Voxel Based Morphometry

### VBM

Voxel based morphometry (VBM) is an automated computerized technique involving iterative steps that include the registration of an individual's brain scan to stereotaxic space, field homogeneity bias correction, and the segmentation of white and gray matter and CSF. Images resulting from VBM pre-processing are stereotaxic “voxel by voxel” maps of tissue volume, which can be used for statistical analyses. The results from studies employing VBM consistently demonstrate atrophy in the medial temporal lobe and the hippocampus in groups of patients with MTLE, compared with controls (Bonilha et al., 2004; Keller et al., 2002). These robust group differences suggest that VBM may be useful for computer-aided detection of atrophy for individual MTLE patients. Individual hippocampal gray matter maps from VBM can be used to detect the presence of HS, when compared with healthy subjects. The use of high-resolution MRI, in combination with modern automated techniques, can improve the sensitivity of automatic HS detection. Further, analysis of MTLE is associated with a clear anatomical hypothesis (e.g. abnormality in the hippocampus) that can be used to maximize statistical power (with focusing on the distribution of signal throughout this region). Hence, a voxel-wise presentation was developed of standardized Z scores for each subject, in comparison with a matched population.

Disclosed herein are methods and systems that provide for automatic detection of brain areas that are abnormally small or large. The diagnosis of many neurological diseases relies on the visual detection of areas in the brain that are abnormally small. In some forms, the methods and systems can map the locations in the brain where there is a difference in brain tissue compared to the normal population. The quantification through the use of Z-scores enables the clinician to gauge how abnormal this area is. Embodiments can assist a clinician when judging images (including mri images).

### D. Methods, Computers and Systems

Disclosed herein are methods of detecting body region abnormality in a subject, comprising the steps of:

a. selecting a body region of clinical concern;

b. imaging the body region;

c. analyzing and assigning the body region through a comparison score; and

d. analyzing the comparison score distribution, wherein high or low comparison scores can indicate body region abnormality.

Also disclosed herein are computer systems, comprising computer components adapted to execute the method comprising the steps of:

a. receiving data from imaging of a body region;

b. analyzing and assigning the body region through a comparison score; and

c. analyzing the comparison score distribution, wherein high or low comparison scores can indicate body region abnormality.

d. outputting the results from c.

Also disclosed herein are methods of identifying a subject with body region abnormalities, comprising the steps of:

a. selecting a body region of clinical concern;

b. imaging the body region;

c. analyzing and assigning the body region through a comparison score; and

d. analyzing the comparison score distribution, wherein high or low comparison scores can indicate body region abnormality.

In some forms of the disclosed methods and computer systems, the comparison score can be a Z score. In some forms, the comparison score can be any comparison with a population standard.

In some forms, a body region can be any body region that that can be imaged. In some forms, the body region can be the brain region, bones region, heart region, vascular system region, lung region or organ region. In some forms, the body region can be the brain region or bone region. In some forms, the body region can be the brain region. In some forms, the brain region can be the hippocampus.

In some forms, the body regions can be selected. In some forms, the body regions can be selected by analyzing symptoms of the subject. In some forms, the body region is a body region that is likely can cause the symptoms. In some forms, the body region is selected by a medical professional.

In some forms analyzing the body region can comprise using a quantitative methodology. In some forms, the quantitative methodology can assign a value to each unit of the image. In some forms, the analyzing the body region can comprises using voxel-based morphometry. In some forms, analyzing the image comprises assigning a comparison score to the image. In some forms, the image is assigned a comparison score. Calculating a comparison score is known in the art. In some forms, the comparison score can be a voxel-wise comparison score. In some forms, the voxel-wise comparison score represents the density of volume of a body region or specific regions within the body region. In some forms, the comparison score can represent gray matter volumes. In some forms, the comparison score can represent, bone density or bone volume. In some forms, the comparison score can be a Z score.

In some forms, the comparison score can be derived from a nation, regional, localized or specific data base. The database can be specific to the imaging instrument. The comparison score can be derived from a combination of any national, regional, localized or specific data base. The population standard can be limited to a specific population. For example, the comparison score can be derived by the population standard for a body region for a specific population. The population can be based on age, gender, race, weight, height, geographical inhabitance, pervious diagnosis of a disease, etc. The comparison score for a subject can be analyzed based on common characteristics between the population and the subject. For example, a child's comparison score can be computed based on the population standard based on children. In some forms, the population standard is based on healthy subjects in a population. In some forms, the population standard is based on the comparison scores from both all members of a population. In some forms, analyzing the comparison score to a population standard can comprise comparing the lowest 2.5%, 5%, 7.5%, 10%, 15%, 20%, 25%, 30%, 35%, 40%, 45%, 50% or any combination thereof of comparison score values. In some forms, analyzing the comparison score to a population standard can comprise comparing the lowest 15%, 20%, 25%, 30% or any combination thereof of comparison values. In some forms the comparison score can be a mean voxel-wise comparison score for a particular location. The mean voxel-wise comparison score, can be based on any limitation disclosed herein.

In some forms the subject can be at risk of having a body region abnormality. In some form the body region abnormality can be a brain region abnormality. For example, the brain region abnormality can be atrophy. In some forms, the subject could be diagnosed with a body region abnormality. In some forms, the subject can have symptoms of a body region abnormality. In some forms, the subject can be monitored for body region abnormalities. In some forms, the subject can be suffering from body region abnormalities. In some forms, the subject can be in need of treatment for body region abnormalities. In some forms, the body region abnormality is a disease. For example, osteoporosis. In some forms, the body region abnormality can cause a disease. For example, hippocampal atrophy can be associated with hippocampal sclerosis. In some forms, subject can be recommended treatment for a disease. In some forms, the disease is MTLE.

Also disclosed herein are machines, apparati, and systems, which are designed to perform the various methods disclosed herein. It is understood that these can be multipurpose machines having modules and/or components dedicated to the performance of the disclosed methods. For example, a imaging instrument can be modified as described herein so that it contains a module and/or component which for example, a) produces a Z-score, and/or performs a imaging analysis, such as a Z-score analysis alone or in any combination. In particular, the modules and components within the imaging instrument or alone can be responsible for determining body region abnormalities. The imaging instrument or alone can be linked to the modules and/or components responsible for analyzing, identifying or detecting comparison score values. Thus, the methods and systems herein can have the data, in any form uploaded by a person operating a device capable of performing the methods disclosed herein.

In addition, or instead, the functionality and approaches discussed above, or portions thereof, can be embodied in instructions executable by a computer, where such instructions are stored in and/or on one or more computer readable storage media. Such media can include primary storage and/or secondary storage integrated with and/or within the computer such as RAM and/or a magnetic disk, and/or separable from the computer such as on a solid state device or removable magnetic or optical disk. The media can use any technology as would be known to those skilled in the art, including, without limitation, ROM, RAM, magnetic, optical, paper, and/or solid state media technology.

In some forms, the method is a computer implemented method. In some forms, the methods disclosed herein can be performed by computers, computer networks, imaging instruments or a combination thereof. In some forms, the imaging of the body region can be done by an imaging instrument. The imaging instrument can for example be X-Ray, electron microscopy, radiographic methods, magnetic resonance imaging (MRI), nuclear medicine, photoacoustic methods, thermal methods, tomography, ultrasound, computed axial tomography, diffuse optical imaging, event-related optical signal, functional magnetic resonance imaging, magnetoencephalography, positron emission tomography or single photon emission computed tomography. In some forms the imaging instrument is a MRI. In some forms, the imaging instrument is adapted to perform the methods described herein. In some forms, the imaging instrument is connected to a computer system or a computer network. In some form the computer system can comprise outputting the results of the methods. In some forms, the outputting of the results can be on a monitor. In some forms, the imaging and images can be acquired such that they are capable of being stored and manipulated in a digital format thus allowing processing and other analysis to take place using microprocessors and/or computers. The computer can be a personal computer. In some forms, conventional personal computer systems as well as other types of processor-based systems can be used to implement the methods and disclosed herein. In some forms, the computer that can be used to implement the methods includes a processor, a system memory, and an input/output (“I/O”) bus. In some forms, a system bus couples the central processing unit to the system memory. In some forms, the bus controller can control the flow of data on the I/O bus and between the central processing unit and a variety of internal and external I/O devices. The I/O devices can be connected to the I/O bus can have direct access to the system memory using a Direct Memory Access (“DMA”) controller.

In some forms, the I/O devices are connected to the I/O bus via a set of device interfaces. The device interfaces can include both hardware components and software components. For example, a hard disk drive and a floppy disk drive for reading or writing removable media may be connected to the I/O bus through disk drive controllers. An optical disk drive for reading or writing optical media can be connected to the I/O bus using a Small Computer System Interface (“SCSI”). Alternatively, an IDE (ATAPI) or EIDE interface can be associated with an optical drive such as can be the case with a CD-ROM drive. The drives and their associated computer-readable media provide nonvolatile storage for the computer. In addition to the computer-readable media described above, other types of computer-readable media may also be used, such as ZIP drives, or the like.

A display device, such as a monitor, is connected to the I/O bus via another interface, such as a video adapter. A parallel interface connects synchronous peripheral devices, such as a laser printer to the I/O bus. A serial interface connects communication devices to the I/O bus. In some forms, a user can enter commands and information to the computer via a serial interface or by using an input device such as a keyboard, mouse, touch screen, or modem. Other peripheral devices may also be connected to the computer, such as audio input/output devices or image capture devices.

In some forms, a number of program modules can be stored on the drives and in the system memory. The system memory can include both Random Access Memory (“RAM”) and Read Only Memory (“ROM”). The program modules control how the computer functions and interacts with the user, with I/O devices, or with other computers. Program modules include routines, operating systems, application programs, data structures, and other software or firmware components. In some forms, the methods can compromise one or more program modules stored on the drives or in the system memory of the computer. Modules may thus comprise computer executable instructions for performing the algorithm steps described herein.

In some forms, the computer can operate in a networked environment using logical connections to one or more remote computers. The remote computer may be a server, a router, a peer device or other common network node, and typically includes all or many of the elements already described for the computer. In a networked environment, program modules and data may be stored on the remote computer. The logical connections include a local area network (“LAN”) and a wide area network (“WAN”). In a LAN environment, a network interface such as an Ethernet adapter card, can be used to connect the computer to the remote computer. In a WAN environment, the computer may use a telecommunications device, such as a modem, to establish a connection. Other connection methods may be used, and networks may include such things as the “world wide web”.

In some forms, the operator can control the personal computer using a keyboard and or a mouse, and receives information on status and results from the monitor. The CPU executes computer software that performs the methods described herein. Embodiments of the invention can be implemented in a computer wherein the statistically acquired “normal” image is saved using typical devices such as magnetic media or electronic storage devices and retrieved to compare to images, including processed images, of the subject of interest.

In some forms the methods can further comprise the step of outputting the results the methods.

The method of claim 1, wherein method is computer implemented in an imaging instrument. In some forms outputting the results from the methods can comprise identifying deviations in the Z-score.

In some forms, the computer or computer network can receive data from an imaging instrument. In some forms, the computer or computer network analyzes and outputs the data from the imaging instruments.

### 1. EXAMPLES

### i. Example 1

Hippocampal sclerosis is frequently associated with hippocampal atrophy (HA), which is often observed on routine MRI of patients with medial temporal lobe epilepsy (MTLE). Manual morphometry of the hippocampus is sensitive to detecting HA, but is time consuming and prone to operator error. Automated MRI morphometry has the potential to provide rapid and accurate assistance in the clinical detection of HA.

a. Methods:

A voxel-based morphometry analysis was performed of 23 consecutive subjects with MTLE and 58 matched controls. Images from randomly selected 34 controls were used to create mean and standard deviation images of gray matter volume. Voxel-wise standardized Z score hippocampal images from patients and the remaining 24 controls were crosschecked with receiver operating characteristic (ROC) curves to evaluate sensitivity versus 1-specificity rate for a binary classifier (atrophied versus normal hippocampi).

b. Results:

The ipsilateral hippocampi of patients with MTLE displayed a significantly lower mean Z score compared to the hippocampi of controls (F(2,67)=33.014, p<0.001, Tukey HSD <0.001). A classifier using the hippocampal gray matter Z scores to discriminate between atrophied and normal hippocampi yielded a fitted ROC=97.3, traditionally considered an excellent discriminator, with a standard error of classification of 1.173 individuals if 100 patients and 100 controls are studied.

c. Conclusion:

Automatic morphometry can be used as a clinical tool to assist the detection of HA in patients with MTLE. It can provide a quantifiable estimative of atrophy, which can aid in the decision about the presence of clinically relevant HA.

### ii. Example 2

### a. Methods

Twenty-three consecutive patients with the clinical and neurophysiological characteristics of MTLE, and visually defined hippocampal atrophy were selected for this study (mean age=38±11 years, 12 women).

Patients were referred from the epilepsy clinic at the Medical University of South Carolina, where they were diagnosed based on comprehensive neurological evaluation, which included a careful medical history, neurological examination, interictal EEG and prolonged video-EEG monitoring. The diagnosis of MTLE was based on the International League Against Epilepsy (ILAE) (Commission on classification and terminology of the International League Against Epilepsy). Seizures were clinically lateralized according to the combination of the data from the neurological examination, interictal and prolonged EEG with seizure onset recording. The data from clinical and electrophysiological evaluations were concordant for all patients, who exhibited only unilateral seizure onset. All patients exhibited unilateral visually defined hippocampal atrophy, ipsilateral to the side of seizure origin. Eight patients had right hippocampal atrophy and 15 left hippocampal atrophy.

Fifty-eight healthy individuals (mean age=33±11 years, 29 women) were also enrolled in the study. These groups were not different in age (t(79)=−1.6, p=0.11) or gender distribution (Yates' Chi(1)=0.005, p=0.94). Since the aim of this study is to discriminate between abnormal and normal hippocampi, special attention was placed on selecting a well matched group of controls, thereby avoiding bias related to a different demographic profile between controls and patients.

The Medical University of South Carolina IRB committee approved this study. All subjects signed an informed consent to participate in this study. Subjects underwent high-resolution MRI in a Philips 3T scanner equipped with a multi-element head coil yielding T1-weighted images with 1 mm isotropic voxels.

Images from randomly selected 34 controls (mean age=33±11 years, 17 women), similar to the patient group in age (t(55)=−1.9, p=0.06) and gender distribution (Yates'Chi(1)=0.012, p=0.91) were used to construct a normalized T1 template and tissue (segmented gray and white matter) priors. An age, gender, and scanner appropriate template and tissue priors were constructed from a group of local controls in order to better represent the demographics of our study population and inhomogeneities of the B0 field of the MRI scanner employed in this study.

Images from the patients, images from remaining control subjects [hitherto referred to as the crosscheck group; 24 subjects, matched to the TLE sample for age (mean age=36.5±8 years, t(55)=−1.9, p=0.51) and gender (12 women, Yates' Chi(1)=0.012, p=0.91)], and from the 34 control subjects (whose images were used to build the template and tissue priors) underwent unified segmentation using the study-specific a priori template images. Pre-processing was composed of iterative spatial normalization, modulation and segmentation of gray and white matter using the VBM5 toolbox (http://dbm.neuro.uni-jena.de/vbm/), employing tissue priors from our study specific template and routines from the software SPM5 (http://www.fil.ion.ucl.ac.uk/spm/software/spm5/). Images were modulated to correct for volume displacement during normalization. Modulated gray matter maps were submitted to a spatial smoothing with an isotropic 10 mm filter (Bonilha et al., 2004).

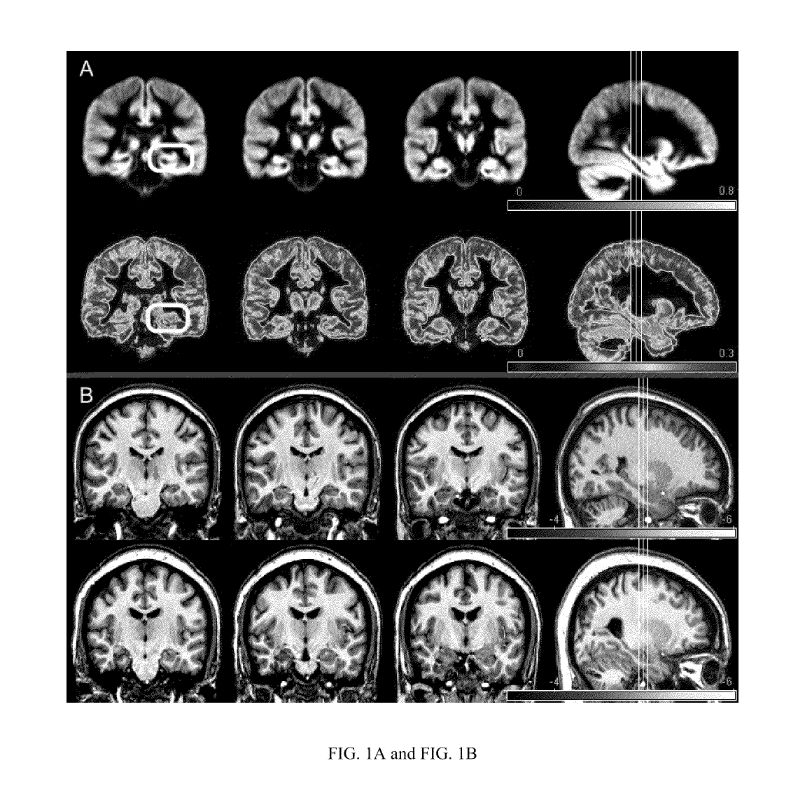

Pre-processed smoothed gray matter maps from controls employed in the construction of the template were used to generate voxel-wise mean and standard deviation maps (FIG. 1), employing the software NPM (http://www.sph.sc.edu/comd/rorden/npm/) (Rorden et al., 2007). The images from the control subjects, which were used to construct the template, were also used to construct mean and standard deviation images. The remaining analyses involved the subjects from the patient and crosscheck groups, i.e., the Z score images from these subjects were calculated based on mean and standard deviation images from an independent group.

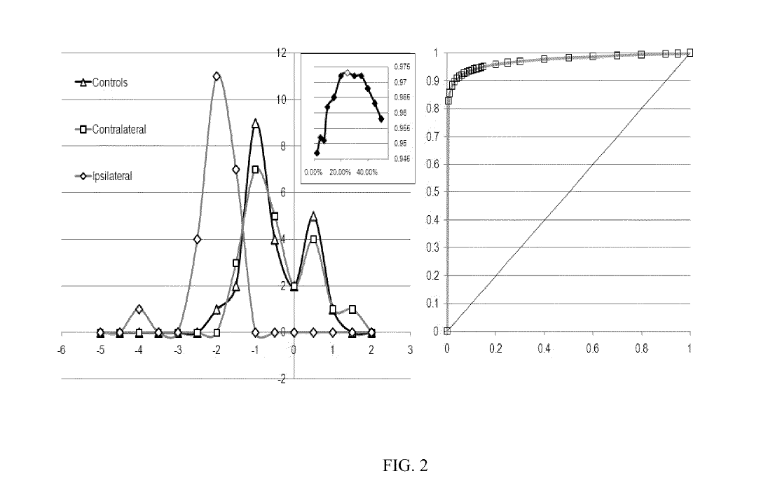

For each patient and for each control subject of the crosscheck group, a voxel-wise map of Z score values, relative to the mean and standard deviation images from control group, was generated. In this Z score map, each voxel represents how many standard deviations the gray matter amount in this voxel for this patient is away from the mean gray matter amount for this same voxel in the control population (FIG. 2).

Subsequent analyses focused on the hippocampal region. A region of interest corresponding to the hippocampus in the Anatomical Automatic Labeling (http://www.cyceron.fr/freeware/) was created, and the Z score of each voxel comprised in hippocampal region of interest was extracted using the Volume toolbox for SPM5 (http://sourceforge.net/projects/spmtools).

The mean voxel-wise Z score was then calculated for each individual, discriminating the mean of sequentially larger groups comprised of the voxels within the lowest 2.5, 5, 7.5, 10, 15, 20, 25, 30, 35, 40, 45 and 50% of the total hippocampal Z score values. For instance, the mean 2.5% Z value corresponded to the mean of the Z scores with the lowest 2.5% in the Z score distribution, and so on. This parametric approach to characterizing damage within the hippocampus was used to increase the sensitivity of the measure and focus on regions that are most affected within the hippocampus rather than diluting the measure of HA by including hippocampal regions that are observed to be relatively unaffected (Bonilha et al., 2004) as demonstrated in the results section.

Mean voxel-wise values (for each percentile described above) were used to construct receiver operating characteristic (ROC) curves to evaluate the sensitivity (true positive rate) versus (1−specificity) (false positive rate) for a binary classifier system (atrophied or normal hippocampi) as its discrimination threshold is varied. The percentile with the highest fitted ROC was then chosen as the best discriminator. The percentile chosen was 25% (FIG. 2—insert). Importantly, only data from the hippocampus ipsilateral to the side of visual atrophy was used to construct ROC curves, as the purpose of this study is to investigate if automatic MRI analysis can discriminate atrophied hippocampi to normal hippocampi. Furthermore, one value was computed for each control subject, and comprised the average of that subject's left and right hippocampi.

Finally, a one-way ANOVA (with three levels: control, side contralateral to hippocampal atrophy and side ipsilateral to atrophy) was computed to assess differences in the mean hippocampal Z score (from the lowest 25% percentile) between patients and the crosscheck control population. Tukey post-hoc test was employed to evaluate differences between groups. The level of statistical significance was set at p<0.05.

### b. Results

The mean of the Z scores comprised within the lowest 25% percentile was observed as the best discriminator between atrophied and normal hippocampi, with the fitted ROC curve=0.973, as shown in FIG. 2 (inset). Nonetheless, the fitted ROC curves for the percentiles between 2.5% and 50% were also good discriminators, with fitted ROC curves ranging from 0.945 (lowest 2.5%) to 0.958 (50%). Larger percentiles were not as good discriminators. The 75% percentile resulted in a fitted ROC=0.687 and the 100% percentile, i.e., the mean of all Z scores within the hippocampi, fitted ROC=0.664. Hence, the remaining results are based on the data corresponding to the mean from the lowest 25%.

The prediction capacity of the Z scores to differentiate normal to atrophied hippocampi is shown in the ROC curve in FIG. 2B. The area under the ROC curve corresponds to 0.973 (i.e., the fitted ROC=97.3), and areas greater than 0.97 are traditionally considered excellent discriminators. According to this distribution, a sensitivity of 91.52% corresponds to a specificity of 95%. Similarly, the predictive power of this distribution yields a standard error of classification of 1.173 individuals if 100 patients and 100 controls are studied.

Confirmatory group comparisons were performed to determine the effect sizes that were associated with the highly sensitive and specific classification results from the ROC analyses above. Among the hippocampi of control subjects, the mean Z score of the lowest 25% was −0.75±1.06 (range −2.14 to 0.83), while in patients with MTLE, the mean Z score of the lowest 25% of the ipsilateral hippocampi was −2.24±0.6 (range −4.34 to −1.5), and of the contralateral hippocampi −0.69±0.8 (range −1.68 to 1.05). The Kolmogorov-Smirnov test confirmed that these samples were normally distributed (controls: KS=0.83, p=0.487; ipsilateral hippocampi: KS=0.89, p=0.404; contralateral hippocampi: KS=0.81, p=0.522). The ipsilateral hippocampi of patients with MTLE displayed a significant lower mean Z score compared to the hippocampi of the control population (F(2,67)=33.014, p<0.001, Tukey HSD <0.001) (FIG. 2A). There was no difference between the contralateral hippocampi and the normal hippocampi (Tukey HSD=0.91), however the ipsilateral hippocampi were significantly lower than the contralateral hippocampi (Tukey HSD <0.001).

c. Discussion

This study aimed to introduce the concept of standardized gray matter maps, i.e., voxel-wise Z scores, as a tool to investigate the presence of hippocampal atrophy. Albeit all patients in this study had visually defined hippocampal atrophy, we observed a high concordance between the visual diagnosis of hippocampal atrophy and the classification based on Z score, as demonstrated by the ROC curve. Specifically, atrophied hippocampi exhibit voxel-wise Z scores underneath a critical Z score threshold. These results show that voxel-wise Z score analyses can aid in the detection of clinically relevant hippocampal atrophy.

As demonstrated by this present study, Z score maps can discriminate atrophied from normal hippocampi. Importantly, the use of Z score maps is an easy to implement and potentially reproducible method, which can be checked and fine-tuned according to patient and control population in each specific center. In particular, the detection of ‘abnormal’ hippocampi depends on a solid definition of what a ‘normal’ hippocampus is. We contend that the definition of normal range of hippocampus gray matter levels depends on three main topics: (1) a large number of control subjects, therefore eliminating the effect of outliers; (2) a control population matched as best as possible to the patient population (so as to avoid bias regarding a different demographic profile); and (3) a similar imaging protocol, performed in the same scanner, for controls and patients, who should be scanned in an a interleaved fashion, thereby avoiding T1 signal changes (if the sequences are not similar) and magnetic field non-homogeneities (if the scanner is not the same, or if the data from the two groups is not collected in an interleaved fashion). This is a preliminary study in which only a limited number of control subjects were used as normative data. The sample size of control and patients used in this example were relatively small. Therefore, even though the predictive power was excellent, it has the potential to be even further improved with larger samples. Increasing the number of subjects in the control population can augment the accuracy of the method, as the resulting smaller confidence interval can facilitate the detection of variations of normality in patients, and flag outliers within the control population.

In this study, the automatic detection of hippocampal atrophy was not compared with other forms of quantification of gray matter within the hippocampus. Importantly, manual morphometry has been consistently shown to increase the likelihood of detection of hippocampal sclerosis on routine MRI (Cendes et al., 1993; Jack, Jr. et al., 1990) compared to visual inspection alone. Since all patients in this study exhibit visual atrophy, they should all also exhibit quantifiable atrophy and manual morphometry would therefore not add to the classification information on the hippocampi. Future work, investigating patients with MTLE without clear-cut visual hippocampal atrophy, could compare the predictive power of both methods. Manual morphometry can be cumbersome and time-consuming, preventing its widespread use in routine clinical practice. Hence, it would be appealing to test if automatic morphometry is comparable, or better, at classifying the presence or absence of brain atrophy.

Voxel-wise Z score maps can also provide additional insight into the pathophysiology of MTLE. The recognition of particular spatial patterns of atrophy within the hippocampus, for instance, disproportionally affecting the hippocampal body as opposed to the head, may provide insight about the nature of hippocampal atrophy related to HS compared to the pattern of hippocampal atrophy as a consequence of seizures. Notably, in this present study we failed to observe a significant difference between the contralateral hippocampus and normal hippocampi. A voxel-wise measure, rather than the composite hippocampal measure used in this study, may have been more sensitive to contralateral atrophy. In addition, this null result may reflect the inclusion of consecutive patients with medial temporal epilepsy and unilateral hippocampal atrophy, with a broad range of atrophy of the contralateral hemisphere, thereby reducing the statistical power regarding the finding abnormalities in the less-affect side.

Furthermore, standardized gray matter maps could also be used to quantify extra-hippocampal gray matter atrophy, thereby enabling a comprehensive structural assessment, which can be useful in the decision making process involving patients with refractory MTLE. As previously demonstrated by our group and others' analyses of MTLE using standardized morphometry (Bonilha et al., 2004; Bonilha et al., 2005; Bonilha et al., 2006; Keller et al., 2002), patients with MTLE demonstrate gray matter atrophy that extends beyond the hippocampus. The detection and quantification of individual extra-hippocampal gray matter atrophy can be potentially useful for predicting cognitive outcome after medical or surgical treatment.

In summary, from the results presented in this manuscript, we show that automatically generated voxel wise gray matter Z score maps of the hippocampi can be used as an additional tool to detect hippocampal atrophy. This is a preliminary study in which the method was outlined, and further validation studies, particularly involving histological confirmation of HS and a larger control population, will lead to identifying a critical Z score threshold that is highly sensitive and specific for hippocampal atrophy and HS. This method will require adjustments regarding different population demographics and MR scanner features, but can be clinically implemented as a quantifiable marker of HS.

### REFERENCES

Blumcke I, Thom M, Wiestler O D (2002). Ammon's horn sclerosis: a maldevelopmental disorder associated with temporal lobe epilepsy. Brain Pathol: 12 199-211.
Bonilha L, Rorden C, Castellano G, Pereira F, Rio P A, Cendes F, Li L M (2004). Voxel-based morphometry reveals gray matter network atrophy in refractory medial temporal lobe epilepsy. Arch Neurol 61: 1379-84.
Bonilha L, Rorden C, Appenzeller S, Coan A C, Cendes F, Li L M (2005). Gray matter atrophy associated with duration of temporal lobe epilepsy. Neuroimage 32: 1070-9.
Bonilha L, Rorden C, Castellano G, Cendes F, Li L M (2006). Voxel-based morphometry of the thalamus in patients with refractory medial temporal lobe epilepsy. Neuroimage 25:1016-21.
Cendes F, Andermann F, Gloor P, Evans A, Jones-Gotman M, Watson C, Melanson D, Olivier A, Peters T, Lopes-Cendes I (1993). MRI volumetric measurement of amygdala and hippocampus in temporal lobe epilepsy. Neurology 43: 719-25.
Commission on classification and terminology of the International League Against Epilepsy. Proposal for revised classification of epilepsies and epileptic syndromes (1989). Epilepsia 30: 389-99.
Jack C R Jr, Sharbrough F W, Twomey C K, Cascino G D, Hirschorn K A, Marsh W R, Zinsmeister A R, Scheithauer B (1990). Temporal lobe seizures: lateralization with MR volume measurements of the hippocampal formation. Radiology 175: 423-9.
Keller S S, Mackay C E, Barrick T R, Wieshmann U C, Howard M A, Roberts N (2002). Voxel-based morphometric comparison of hippocampal and extrahippocampal abnormalities in patients with left and right hippocampal atrophy. Neuroimage 16: 23-31.
Margerison J H, Corsellis J A (1996). Epilepsy and the temporal lobes. A clinical, electroencephalographic and neuropathological study of the brain in epilepsy, with particular reference to the temporal lobes. Brain 89: 499-530.
Rorden C, Bonilha L, Nichols T E (2007). Rank-order versus mean based statistics for neuroimaging. Neuroimage 35: 1531-7.
Sommer W (1880). Erkrankung des Ammonshorns als aetiologisches Moment der Epilepsie. Arch Psychiatr Nervenkr 10: 631-657.

We claim:

1. A method of detecting body region abnormality in a subject, comprising the steps of:
a. selecting a body region of clinical concern;
b. imaging the body region;
c. analyzing and assigning the body region through a comparison score; and
d. analyzing the comparison score distribution, wherein high or low comparison scores indicate body region abnormality.

2. The method of claim 1, wherein the body region is a brain region.

3. The method of claim 1, wherein the brain region is the hippocampus.

4. The method of claim 1, wherein the imaging the body region is performed by X-Ray, electron microscopy, radiographic methods, magnetic resonance imaging (MRI), nuclear medicine, photoacoustic methods, thermal methods, tomography, ultrasound, computed axial tomography, diffuse optical imaging, event-related optical signal, functional magnetic resonance imaging, magnetoencephalography, positron emission tomography or single photon emission computed tomography.

5. The method of claim 1, wherein analyzing the body region comprises using voxel-based morphometry.

6. The method of claim 1, wherein analyzing the comparison score comprises comparing the lowest 2.5%, 5%, 7.5%, 10%, 15%, 20%, 25%, 30%, 35%, 40%, 45% or 50% or any combination thereof of comparison scores.

7. The method of claim 1, wherein the comparison score and population standards is the mean voxel-wise comparison score.

8. The method of claim 1, wherein the subject is at risk of having body region abnormality.

9. The method of claim 1, wherein the subject has symptoms of body region abnormality.

10. The method of claim 1, wherein the subject has been diagnosed with body region abnormality.

11. The method of claim 1, further comprising diagnosing the subject with a disease associated with the body region abnormality.

12. The method of claim 11, further comprising recommending treatment for the disease.

13. The method of claim 11, wherein the disease is MTLE.

14. The method of claim 1, wherein the body region abnormality is related to atrophy.

15. The method of claim 1, wherein the body region abnormality is hippocampal atrophy.

16. The method of claim 1, wherein the method is a computer implemented method.

17. The method of claim 16, further comprising the step of outputting the results from claim 16.

18. The method of claim 1, wherein method is computer implemented in an imaging instrument.

19. A computer system, comprising computer components adapted to execute the method comprising the steps of:
a. receiving data from imaging of a body region;
b. analyzing and assigning the body region a comparison score compared to a population standard;
c. producing an image of the comparison scores for each voxel, generating an image that the clinician can evaluate and or overlap with the original images, looking for comparison score values at that body part;
d. outputting the results from c.

20. The computer system of claim 19, wherein outputting the results from step c comprises identifying comparison score deviations.

21. The method of claim 1, wherein the comparison score is a Z score.

22. The computer system of claim 19, wherein the comparison score is a Z score.
